(19) United States
(12) Patent Application Publication
(10) Pub. No.: US 2015/0190107 A1
(43) Pub. Date: Jul. 9, 2015

(54) APPARATUS FOR GENERATING IMAGE AND CONTROL METHOD THEREOF

(71) Applicant: Samsung Electronics Co., Ltd., Gyeonggi-do (KR)

(72) Inventors: Hei Soog KIM, Gyeonggi-do (KR); Praveen GULAKA, Gyeonggi-do (KR)

(21) Appl. No.: 14/451,749

(22) Filed: Aug. 5, 2014

(30) Foreign Application Priority Data
Jan. 8, 2014 (KR) 10-2014-0002548

(51) Int. Cl. A61B 6/00; A61B 5/00; A61B 5/055

(52) U.S. Cl. CPC A61B 6/5235; A61B 5/055; A61B 5/0073

(57) ABSTRACT

An apparatus for generating an diagnostic image of an object including a first image capturing unit to acquire a first diagnostic image, a second diagnostic image capturing unit that captures images by utilizing a different format of image capturing than the first image capturing unit in order to acquire a second diagnostic image that is different from the first diagnostic image. An image processor is configured to extract distinct points from at least one of the first diagnostic image and the second diagnostic image and perform image registration the first and second diagnostic images based on the extracted distinct points.

### CLAIM OF PRIORITY

This application claims the benefit of Korean Patent Application No. 10-2014-0002548, filed on Jan. 8, 2014 in the Korean Intellectual Property Office, the disclosure of which is incorporated herein by reference in its entirety.

### BACKGROUND

1. Technical Field

Embodiments of the present disclosure relate to apparatuses for generating a diagnostic image by performing image registration of a plurality of diagnostic images and methods of controlling the same.

2. Description of the Related Art

In general, a medical imaging apparatus is an apparatus that provides a diagnostic image with respect to a patient by acquiring information of the patient. Examples of various types of medical imaging apparatuses include an X-ray imaging apparatus, ultrasonic diagnostic apparatus, a computed tomography (CT) scanner, a magnetic resonance imaging (MRI) apparatus, and the like, to name some non-limiting possibilities.

However, each of the above-mentioned medical imaging apparatuses has advantages and disadvantages. For example, although an MRI apparatus does not use radiation, and acquires a diagnostic image under less strict conditions creates a high contrast image from soft tissues, and provides various diagnostic images, MRI is a relatively slow and expensive. In addition, although CT scanning is relatively quick and inexpensive, a CT scanner cannot provide a high resolution diagnostic image, and a patient is exposed to radiation.

### SUMMARY

Therefore, it is an aspect of the present invention to provide an apparatus for generating a diagnostic image of an object by performing image registration of a plurality of diagnostic images with respect to the object as acquired by using various different methods of image generation and a method of controlling the same.

Additional aspects of the invention will be set forth in part in the description which follows and, in part, will be enabled to a person of ordinary skill from the description, or may be learned by practice of the invention.

In accordance with one aspect of the present invention, an apparatus for generating a diagnostic image includes a first image capturing unit to acquire a first diagnostic image, a second image capturing unit that utilizes a different type of image capturing than the first image capturing unit to acquire a second diagnostic image that is different (from the first diagnostic image, and an image processor configured to extract distinct points from at least one of the first diagnostic image and the second diagnostic image and perform image registration of the first and second diagnostic images based on the extracted distinct points.

The image processor, which comprises hardware such as an integrated circuit, may extract the distinct points from at least one of the first diagnostic image and the second diagnostic image based on pre-stored anatomical information. The image processor may be configured to determine anatomical characteristics to be used as references for image registration by analyzing locations of the first and second diagnostic images, and extract the distinct points by comparing the determined anatomical characteristics with the pre-stored anatomical information.

The image processor may be configured to identify the locations of the first and second diagnostic images by comparing the pre-stored anatomical information with the first and second diagnostic images. The image processor may be configured to identify the locations of the first and second diagnostic images based on image capture conditions for the first and second diagnostic images.

The first and second diagnostic images may include an object and markers attached to the object, and the image processor identifies and extracts the distinct points based on locations of the markers.

The image processor may be configured to normalize at least one of the first and second diagnostic images.

The image processor may also be configured to determine (i.e. assign) weights for the first and second diagnostic images, and perform image registration of the first and second diagnostic images by using the weights. The image processor may determine the weights according to respective types of the first image capturing unit and the second image capturing unit.

The first or second image capturing unit may include at least one apparatus including but not limited to a magnetic resonance imaging (MRI) apparatus, a computed tomography (CT) scanner, a positron emission tomography (PET) scanner, a single photon emission computed tomography (SPECT) scanner, and a mammography apparatus, just to name a few non-limiting possibilities of the different types of image capturing units.

In accordance with another aspect of the present invention, a method of controlling an apparatus for generating a diagnostic image including acquiring a first diagnostic image by a first image capturing unit, and acquiring a second diagnostic image different from the first diagnostic image by a second image capturing unit that utilizes a different type of image capturing than the first image capturing unit, extracting distinct points from the first diagnostic image and the second diagnostic image, and performing image registration of the first and second diagnostic images based on the extracted distinct points. It is to be understood and appreciated by an artisan that the terms such as “a second image capturing unit utilizing a different format of image capturing than the first image capturing unit and being configured to acquire a second image different from the first image” can mean that, for example, the first image capturing unit is a CT scanner, and the second imaging capturing unit is, for example, an MRI machine. Thus a different format will generate different a second diagnostic image that is different than the first diagnostic image.

The extracting of the distinct points may include, for example, extracting the distinct points based on pre-stored anatomical information.

The extracting of the distinct points may include, for example, analyzing locations of the first and second diagnostic images, determining anatomical characteristics to be used as references for image registration based on the identified locations of the first and second diagnostic images, and extracting the distinct points by comparing the determined anatomical characteristics with the pre-stored anatomical information.

The identified locations of the first and second diagnostic images may be identified, for example, by comparing the pre-stored anatomical information with the first and second diagnostic images. The locations of the first and second diagnostic images may be identified based on image capture conditions for the first and second diagnostic images.

The acquiring of the first and second diagnostic images may include, for example, acquiring the first diagnostic image and the second diagnostic image by simultaneously capturing images of the object and at least one marker, and the extracting of the distinct points is performed by extracting the distinct points based on locations of the markers.

The performing image registration of the first and second diagnostic images may include determining weights for the first and second diagnostic images, and processing overlapped portions of the first and second diagnostic images according to the determined weights.

The method may further include normalizing at least one of the first and second diagnostic images.

The first or second image capturing unit may include at least one apparatus including but not limited to a magnetic resonance imaging (MRI) apparatus, a computed tomography (CT) scanner, a positron emission tomography (PET) scanner, a single photon emission computed tomography (SPECT) scanner, and a mammography apparatus.

### BRIEF DESCRIPTION OF THE DRAWINGS

These and/or other aspects of the invention will become apparent and more readily appreciated by a person of ordinary skill in the art from the following description of the embodiments, taken in conjunction with the accompanying drawings of which:

FIG. 1 is a conceptual diagram illustrating an apparatus for generating a diagnostic image according to an embodiment of the present invention;

FIG. 2 is a control block diagram for describing an apparatus for generating a diagnostic image according to an embodiment of the present invention in more detail;

FIG. 3 is a perspective view illustrating an appearance of a first image capturing unit of an apparatus for generating a diagnostic image according to an embodiment of the present invention;

FIG. 4 is a cross-sectional view schematically illustrating a radiation source to emit X-rays of FIG. 3;

FIG. 5 is a schematic diagram illustrating a radiation detector to detect X-rays of FIG. 3;

FIG. 6 is a schematic diagram illustrating an appearance of a second image capturing unit;

FIG. 7 is a diagram illustrating a bore structure and a gradient coil unit of FIG. 6;

FIG. 8A, FIG. 8B and FIG. 8C are diagrams illustrating an example of image registration, in which FIG. 8A illustrates a first diagnostic image acquired by the first image capturing unit 100, FIG. 8B illustrates a second diagnostic image acquired by the second image capturing unit 200, and FIG. 8C illustrates a registered image according to an embodiment of the present invention;

FIG. 9A, FIG. 9B and FIG. 9C are diagrams illustrating another example of image registration in which FIG. 9A illustrates a first diagnostic image acquired by the first image capturing unit 100, FIG. 9B illustrates a second diagnostic image acquired by the second image capturing unit 200, and FIG. 9C illustrates a registered diagnostic image according to an embodiment of the present invention;

FIG. 10 is a diagram illustrating an example of collecting additional information using markers;

FIG. 11 is a flowchart providing an overview of a method of controlling an apparatus for generating an image according to an embodiment of the present invention; and

FIG. 12 providing an overview for describing a method of extracting registration information according to an embodiment of the present invention.

### DETAILED DESCRIPTION

Reference will now be made in more detail to the aspects of the present invention, examples of which are illustrated in the accompanying drawings, wherein like reference numerals refer to like elements throughout.

The claimed invention will now be described more fully with reference to the accompanying drawings, in which exemplary embodiments of the invention are shown. The claimed invention may, however, be embodied in many different forms and the claims should not be construed as being limited to only the embodiments set forth herein; rather, these embodiments are provided so that this disclosure will be thorough and complete in terms of enablement and written description, and will fully convey the concept of the claimed invention to those of ordinary skill in the art. Like reference numerals denote like elements.

The terms used in the specification will be described briefly, and then embodiments will be described in detail.

The terms used in this specification are selected from currently widely used general terms in consideration of functions and structure of the present invention, but may vary according to the intentions or practices of those skilled in the art or the advent of new technology. Additionally, in certain cases, there may be terms that an applicant may arbitrarily select, and in this case, their meanings are described below. Accordingly, the terms used in this specification should be interpreted on the basis of substantial implications that the terms have and the contents across this specification not the simple names of the terms.

Throughout the specification, the term “comprising” an element does not preclude the other elements but further includes an element unless otherwise stated. In addition, the term “unit” as used herein, refers to, but is not limited to, hardware comprising a software component, or a hardware component such as a FPGA or ASIC that performs certain tasks. A unit may be configured to reside on the addressable storage medium and configured to execute on one or more processors, or microprocessors, the processors and/or microprocessors being comprised of integrated circuitry. Thus, a unit may include, in addition to hardware such as integrated circuitry, by way of example, software components, object-oriented software components, class components and task components, processes, functions, attributes, procedures, subroutines, segments of program code, drivers, firmware, microcode, circuitry, data, databases, data structures, tables, arrays, and variables. The functionality provided for in the components and units may be combined into fewer components and units or further separated into additional components and units, but for the claims the term unit is to be interpreted as including, as the appended claims are not software or software per se.

Hereinafter, the present invention will be described in more detail herein through a discussion of certain exemplary embodiments of the invention with reference to the attached drawings. However, this discussion does not limit the presently claimed invention to particular modes of practice. In the drawings, parts unrelated to the descriptions may be omitted for a clearer description of the claimed invention.

As used herein, the term “image” and “diagnostic image” refers to multi-dimensional data composed of discrete image elements (e.g., pixels for two-dimensional (2D) images and voxels for three-dimensional (3D) images). For example, the diagnostic image may include a medical image of an “object ob” acquired by computed tomography (CT) or magnetic resonance imaging (MRI).

As used herein, the term “object ob” refers to human or animal or a part of or the entire body human or animal. For example, the object ob may include organs such as liver, heart, uterus, brain, breast, and abdomen or blood vessels. In addition, the object ob may also include a “phantom”. A phantom refers to a material that has density, effective atomic number, and volume similar to biological tissues, and may include a spherical phantom having physical properties similar to the human body.

As used herein, the term “user” typically refers to medical professionals such as doctors, nurses, medical laboratory technologists, medical imaging professionals, medical equipment technicians, and the like, without being limited thereto.

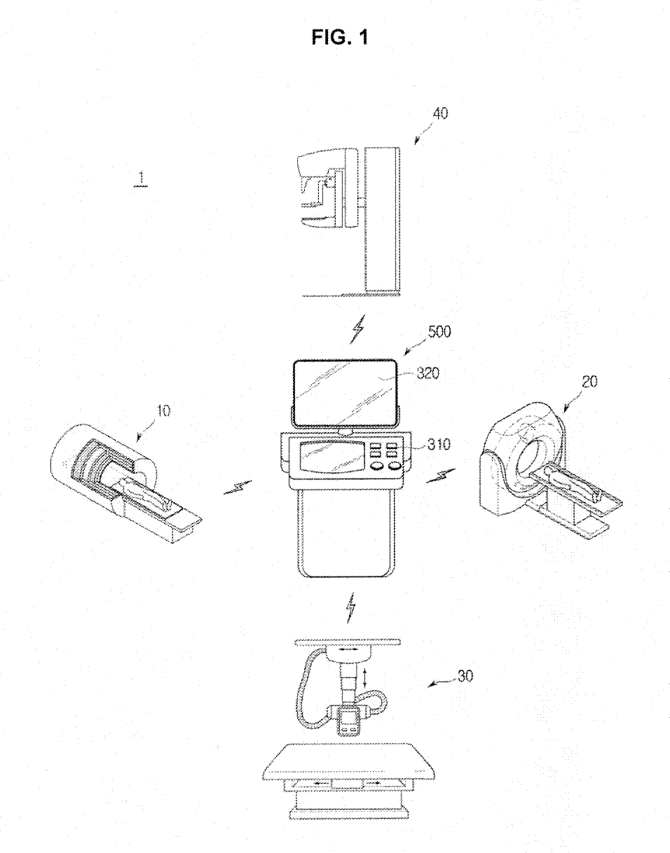

FIG. 1 is a conceptual diagram illustrating an apparatus 1 for generating a diagnostic image according to an embodiment of the present invention.

Referring now to FIG. 1, the image generating apparatus 1 includes a plurality of image capturing units 10, 20, 30, and 40 of different formats to capture internal images of an object ob, and a host device 500 to perform image registration of images received from one or more of the image capturing units 10, 20, 30, and 40.

As illustrated in FIG. 1, the image capturing units 10, 20, 30, and 40 may respectively be spaced at predetermined distances apart from the host device 50 of the image generating apparatus 1. The image capturing units 10, 20, 30, and 40 may respectively be connected to the host device 50 via various wired or wireless communication protocols.

For example, the image capturing units 10, 20, 30, and 40 may respectively perform data communications with the host device 50 via the digital imaging and communications in medicine (DICOM) standard, without being limited thereto. Also, the image capturing units 10, 20, 30, and 40 may respectively communicate with the host device 50 via mobile communication protocols such as the global system for mobile communication (GSM), the code division multiple access (CDMA), the wideband code division multiple access (WCDMA), the time division multiple access (TDMA), and the long term evolution (LTE), just to name a few non-limiting possibilities, and local area network (LAN) protocols such as the wireless local access network (WLAN), Bluetooth, Zigbee, and near field communications (NFC).

In this regard, each of the image capturing units 10, 20, 30, and 40 acquires internal diagnostic images of the object ob by using, for example, radiation, magnetic resonance, or ultrasonic waves. For example, the image capturing units 10, 20, 30, and 40 may acquire internal images of the object ob by using radiation in a same manner as a computed tomography (CT) scanner, a positron emission tomography (PET) scanner, a single photon emission computed tomography (SPECT) scanner, and a mammography apparatus. In addition, the image capturing units 10, 20, 30, and 40 may acquire internal images of the object ob by using magnetic resonance in the same manner as a magnetic resonance imaging (MRI) apparatus or may acquire ultrasonic images by using ultrasonic waves.

As described above, the image capturing units 10, 20, 30, and 40 may acquire diagnostic images of the object ob by using various methods (i.e. formats, types). Each of the methods of acquiring diagnostic images has advantages and disadvantages. For example, while CT is relatively quick and inexpensive, MRI is relatively slow and expensive. However, a higher resolution diagnostic image may be obtained by MRI than by CT.

In addition, images may be acquired by using different methods according to the inner structure and characteristics of the object ob. For example, if the object ob is a human body, diagnostic images for diagnosing diseases may be acquired by using different methods according to structures or characteristics of organs. Thus, diagnosis may more efficiently be performed by acquiring diagnostic images by using suitable methods on the basis of organs and registering the acquired images. In addition, because the diagnostic images are acquired by using suitable methods selected according to the organs, time and costs for acquiring a diagnostic image may be reduced.

Hereinafter, an example of generating a diagnostic image by using CT scanning and MRI will be described for descriptive convenience. However, embodiments of the present invention are not limited thereto, the CT scanning and MRI may be substituted or modified by another method of acquiring inner images of the body or object. Furthermore, diagnostic images may be generated by applying various other methods.

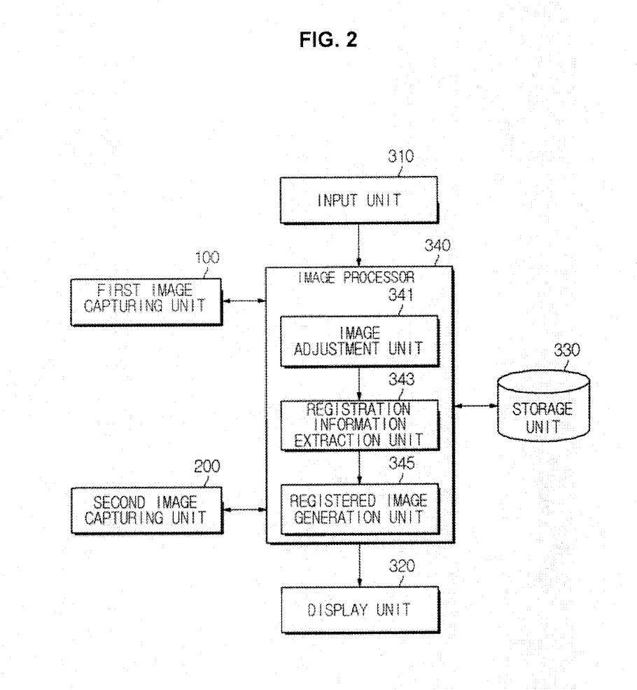

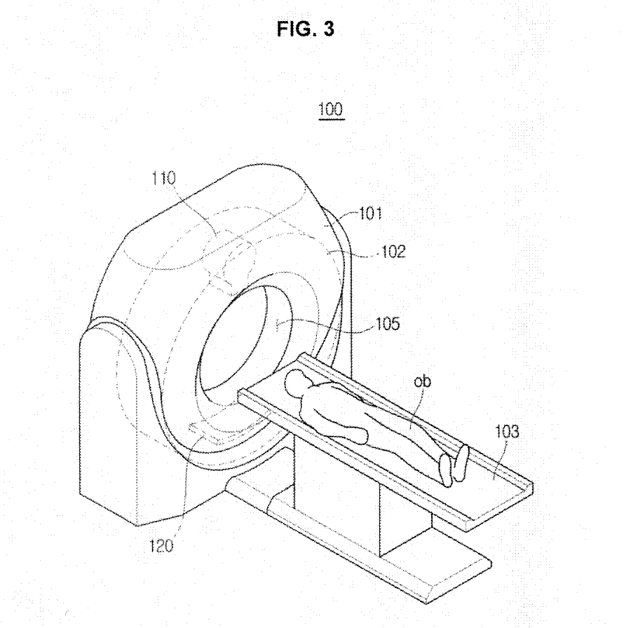

FIG. 2 is a control block diagram for describing an apparatus 1 for generating a diagnostic image according to an embodiment of the present invention in detail.

Referring now to FIG. 2, the image generating apparatus 1 may include a first image capturing unit 100 to acquire a first diagnostic image, a second image capturing unit 200 to acquire a second diagnostic image, an input unit 310 to receive a control instruction from a user, a display unit 320 to display various information, a storage unit 330 comprising non-transitory machine readable medium to store information required to drive the image generating apparatus 1, and an image processor/controller 340 to generate a registered diagnostic image through the overall control of the image generating apparatus 1. As the claimed invention is statutory and not software per se, all of the units shown in FIG. 2 comprise hardware that may be configured for operation.

In this regard, the registered diagnostic image is an image generated by performing image registration of diagnostic images of the object ob acquired by a plurality of image capturing units 100 and 200. In order to generate the registered diagnostic image, captured images need to be acquired.

Hereinafter, the first image capturing unit 100 will be described in more detail with reference to FIGS. 3 to 6.

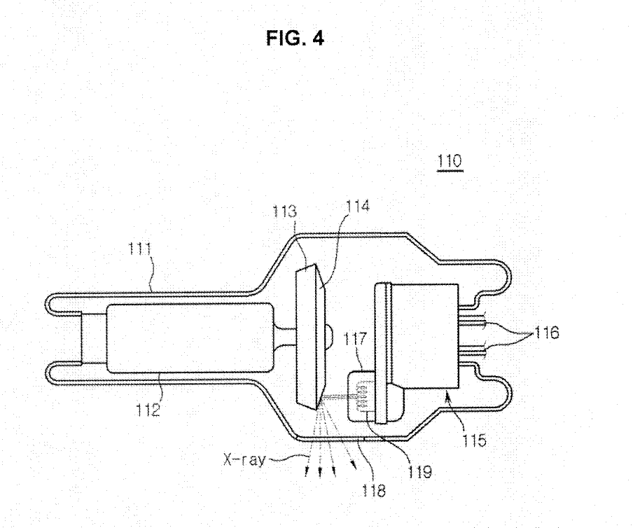

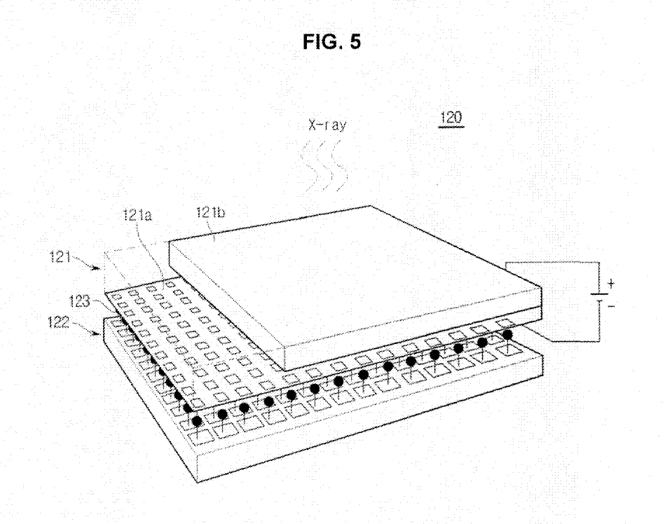

FIG. 3 is a perspective view illustrating one way a first image capturing unit 100 of the image generating apparatus may be constructed. FIG. 4 is a cross-sectional view schematically illustrating a radiation source 110 to emit X-rays of FIG. 3. FIG. 5 is a schematic diagram illustrating a radiation detector 120 to detect X-rays of FIG. 3.

As illustrated in FIG. 3, the first image capturing unit 100 may include a radiation source 110 to emit radiation to the object ob and a radiation detector 120 to detect radiation passing through the object ob. The radiation source 110 and the radiation detector 120 are mounted on a gantry 102 so as to be facing each other, and the gantry 102 is mounted in a housing 101.

When a patient table 103 on which the object ob lies down is moved into the bore 105, diagnostic images of the object ob are captured while the gantry 102 provided with the radiation source 110 and the radiation detector 120 rotates 360 degrees around the bore 105. As a result, at least one first diagnostic image is acquired via radiation.

The radiation source 110 may emit X-rays, gamma rays, alpha rays, beta rays, or neutron rays. For example, the radiation source 110 of the first diagnostic image capturing unit 100 may emit X-rays.

Particularly, the X-ray source 110 may include a bipolar X-ray tube including a positive electrode 113 and a negative electrode 115 as illustrated in FIG. 4 and generate X-rays. The negative electrode 115 includes a filament 119 and a focusing electrode 117, which focuses electrons. The focusing electrode 117 is also referred to by those of ordinary skill as a focusing cup.

Thermoelectrons are generated by maintaining the inside of a glass tube 111 in a high vacuum state of about 10 mmHg and heating the filament 119 of the negative electrode 115. The filament 119 may be a tungsten filament and may be heated by supplying current into an electric wire 116 connected to the filament 119.

With continued reference to FIG. 4, the positive electrode 113 is mainly formed of copper, and a target material 114 is coated or disposed at a portion facing the negative electrode 115. The target material 114 may be a high-resistant material such as Cr, Fe, Co, Ni, W, and Mo. As a melting point of the target material 114 increases, a focal spot size decreases. Here, the focus refers to an effective focal spot. In addition, the target material 114 is inclined by a predetermined degree. As the slope decreases, the focal spot size decreases.

In addition, when a high voltage is applied between the negative electrode 115 and the positive electrode 113, generation of thermoelectrons is accelerated, and the generated thermoelectrons collide with the target material 114 of the positive electrode 113, thereby generating X-rays. The generated X-rays are emitted to the outside through a window 118, and the window 118 may be a beryllium (Be) thin film. In this case, X-rays of a predetermined energy band may be filtered by locating a filter at a front or rear surface of the window 118.

The target material 114 may be rotated by a rotor 112. When the target material 114 is rotated, heat accumulation rate is increased by 10 times or more compared to when the target material 114 is fixed, and the focal spot size is decreased.

The voltage applied between the negative electrode 115 and the positive electrode 113 of the X-ray source 110 is referred to as a “tube voltage”, and an intensity of the tube voltage may be represented as a wave-height (kvp). As the tube voltage increases, the thermoelectrons increase in speed. As a result, energy of X-rays (photon energy) generated as the thermoelectrons collide with the target material 114 increases. Current flowing in the X-ray source 110 is referred to as “tube current” and may be represented as an average value (mA). As the tube current increases, the number of thermoelectrons emitted from the filament increases. As a result, a dose of X-rays (number of X-ray photons) generated as the thermoelectrons collide with the target material 114 increases.

Thus, since the energy of X-rays may be controlled by the tube voltage, and the intensity or dose of X-rays may be controlled by the tube current and exposure time of X-rays, the energy and intensity of X-rays may be controlled according to types or characteristics of the object ob.

In a case where the emitted X-rays have a particular energy band, the energy band may be defined by using an upper limit and a lower limit thereof. The upper limit of the energy band, i.e., maximum energy of the emitted X-rays, may be controlled by the intensity of the tube voltage. Conversely, the lower limit of the energy band, i.e., minimum energy of the emitted X-rays, may be controlled by the filter. When X-rays of a low energy band are filtered by using the filter, average energy of the emitted X-rays may be increased.

As illustrated in detail FIG. 5, the radiation detector 120 (as in FIG. 1) detects X-rays passing through the object ob and outputs a detection signal. In computed tomography, the radiation detector 120 is also called a data acquisition system (DAS). The radiation detector 120 may include a plurality of detectors mounted on a frame in a one-dimensional array.

Particularly, the radiation detector 120 may include a light receiving device 121 that detects X-rays and converts the X-rays into electric signals and a readout circuit 122 that reads the electric signals from the light receiving device 121. In this regard, the readout circuit 122 has a 2D pixel array including a plurality of pixels. The light receiving device 121 may be formed of a single crystal semiconductor material to obtain a high resolution, quick response time, and high dynamic range at low energy and low dose of X-rays. The single crystal semiconductor material may include Ge, CdTe, CdZnTe, GaAs, and the like, just to name a few non-limiting possibilities.

With continued reference to FIG. 5, the light receiving device 121 of radiation detector 120 may have a PIN photodiode structure by bonding a p-type semiconductor layer 121a, in which a plurality of p-type semiconductors are aligned in a 2D pixel array, to the lower surface of the highly resistant n-type semiconductor substrate 121b. A CMOS readout circuit 122 is connected to the light receiving device 121 on the pixel basis. The CMOS readout circuit 122 and the light receiving device 121 may be connected to each other by flip chip bonding, which includes forming a bump 123 using PbSn, In, and the like, reflowing, and pressing the structure while heating. However, the aforementioned structure is discussed as an example of the radiation detector 120, and the structure of the radiation detector 120 is not limited thereto.

The first image capturing unit 100 transmits a first diagnostic image acquired as described above to an image processor. In this regard, the first diagnostic image may include a detection signal output from the radiation detector 120 and information image capture conditions, such as tube voltage and image capturing angle.

The first diagnostic image may also include data obtained by preprocessing the detection signal output from the radiation detector 120 or from a cross-sectional image of the object ob formed based on the detection signal and image capture conditions. In this regard, the preprocessing includes, for example, the correction of unevenness in sensitivity of pixels and the correction of extreme lowering of a signal intensity or loss of a signal mainly due to an X-ray absorber such as a metal.

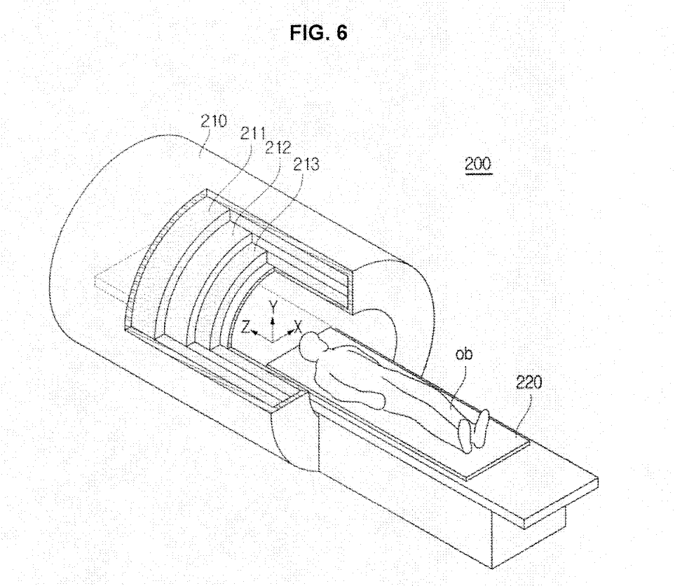

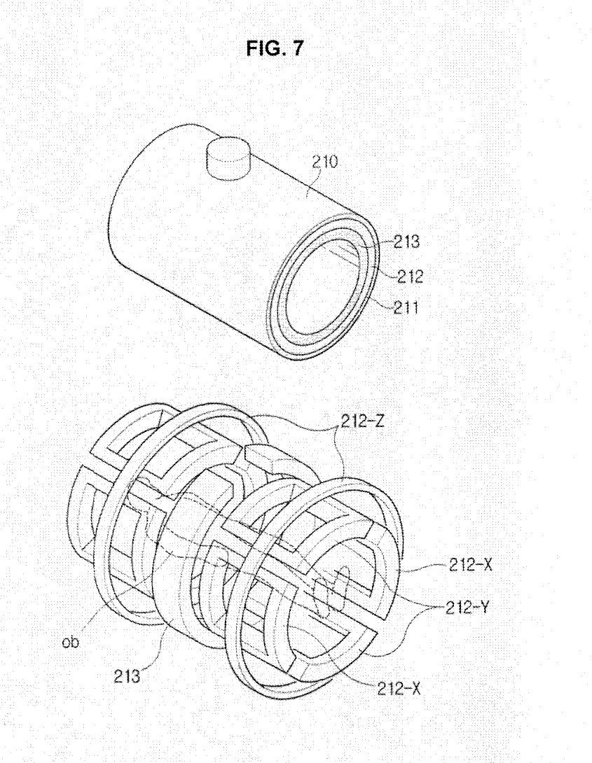

Hereinafter, the second image capturing unit 200 will be described in more detail with reference to FIGS. 6 and 7.

FIG. 6 is a schematic diagram illustrating an appearance of the second image capturing unit 200. FIG. 7 is a diagram illustrating a bore structure and a gradient coil unit of FIG. 6.

The second image capturing unit 200 may acquire a diagnostic image of the object ob by expressing an intensity of magnetic resonance (MR) signals, with respect to radio frequency (RF) signals, generated in a magnetic field having a predetermined intensity by using contrast. For example, when the object ob is lying down in a strong magnetic field and is momentarily irradiated with RF signals, which make nuclei of particular atoms, e.g., hydrogen nuclei, and the irradiation of the RF signals is stopped, MR signals are emitted from the nuclei. An MRI system receives the MR signals to acquire an MR image. The MR signal refers to an RF signal radiated from the object ob.

In this regard, the intensity of the MR signal may be determined by a concentration of a predetermined atom, e.g., hydrogen, contained in the object ob, a relaxation time T1, a relaxation time T2, and a flow such as blood stream.

Particularly, an artisan should appreciate that the second image capturing unit 200 may include a bore 210 that forms a magnetic field and generates resonance with respect to atomic nuclei so as to generate an diagnostic image by supplying a constant frequency and energy to the atomic nuclei, to which a predetermined magnetic field is applied, and converting energy output from the atomic nuclei into a signal.

Referring to FIG. 7, the bore 210 has a cylindrical shape with a hollow inside, and the inner space of the bore 210 is referred to as a cavity. A transport unit 220 transports the object ob lying down thereon into the cavity to receive the MR signal.

In addition, the bore 210 may include a static coil unit 211 that forms a static magnetic field, a gradient coil unit 212 that forms a gradient magnetic field in the static magnetic field, and an RF coil unit 213 that excites atomic nuclei by applying RF pulses thereto and receives echo signals from the atomic nuclei.

The static coil unit 211 may have a shape in which coils surround the cavity. When current is supplied into the static coil unit 211, a static field is formed in the bore 210, i.e., in the cavity. In this case, the static field may generally be formed parallel to a coaxial line of the bore 210. As the static coil unit 211 forms a stronger and more uniform static field, a more precise and accurate diagnostic image of the object ob may be acquired.

When the static field is formed in the cavity, the nuclei of atoms constituting the object ob, particularly, hydrogen atoms, are aligned in the direction of the static field and move around the field axis in a motion known as “precession”. For example, protons have a precession frequency of 42.58 MHZ in an external magnetic field of 1 T. Since hydrogen accounts for the largest portion among atoms constituting a human body, an MRI apparatus may generally acquire MR signals by using a precession of protons.

The gradient coil unit 212 generates a gradient in the static field formed in the cavity to generate a gradient magnetic field. In this case, in order to obtain 3D spatial information, gradient magnetic fields need to be formed in x, y, and z axes.

Accordingly, the gradient coil unit 212 includes three pairs of gradient coils for forming such gradient magnetic fields.

As illustrated in FIG. 7, z-axial gradient coils 212_z generally include a pair of ring-shaped coils, and y-axial gradient coils 212_y are disposed at upper and lower positions of the object ob. X-axial gradient coils 212_x may be disposed at both sides of the object ob.

In this regard, the gradient coil unit 212 may collect the location information of portions of the object ob by differently inducing resonance frequencies in these portions.

The RF coil unit 213 may transmit RF signals to a patient and receive MR signals from the patient. Particularly, the RF coil unit 213 may transmit RF signals having the same frequency as that of precession toward the atomic nuclei in precession, stop the transmitting of the RF signals, and then receive MR signals from the patient.

For example, the RF coil unit 213 may generate electromagnetic signals having a radio frequency corresponding to the type of atomic nuclei, e.g., RF signals, and transmit the electromagnetic signals to the object ob in order to transit an energy level of the nuclei from a lower state to a higher state. When the electromagnetic signals generated by the RF coil unit 213 are applied to the atomic nuclei, energy transition may occur in the nuclei from the lower energy state to the higher energy state. Then, when the electromagnetic waves generated by the RF coil unit 213 disappear, electromagnetic waves having a Larmor frequency may be emitted from the nuclei while the nuclei drop to the lower energy state from the higher energy state. In other words, when the application of the electromagnetic waves to the nuclei is stopped, electromagnetic waves having a Larmor frequency may be emitted as the energy level of the nuclei is transmitted from the higher energy state to the lower energy state. The RF coil unit 213 may receive electromagnetic signals output from the nuclei in the object ob.

The RF coil unit 213 may be implemented as a single RF transceiver coil that serves both as a transmitter to generate electromagnetic waves having a radio frequency corresponding to the type of atomic nuclei and a receiver to receive the electromagnetic waves radiated from the atomic nuclei. In addition, the RF coil unit 213 may also be implemented separately using an RF transmitter coil to generate electromagnetic waves having a radio frequency corresponding to the type of the atomic nuclei and a RF receiver coil to receive the electromagnetic waves radiated from the atomic nuclei.

In addition, the RF coil unit 213 may be fixed to the gantry 20 as illustrated in FIG. 7 or may be detachably mounted thereon. The detachable RF coil unit 213 may include RF coils of portions of the object ob including a head RF coil, a chest RF coil, a leg RF coil, a neck RF coil, a shoulder RF coil, a wrist RF coil, and an ankle RF coil, and the like.

The second diagnostic image capturing unit 200 transmits a second diagnostic image acquired as described above to the image processor. In this regard, the second diagnostic image may include MR signals received by the RF coil unit 213 and diagnostic image capture conditions, such as image capture angle and location of the object ob.

The second diagnostic image may also include data obtained by preprocessing the MR signals received by the RF coil unit 213 or a diagnostic image generated based on the MR signals and image capture conditions. In this regard, the preprocessing of the MR signals may include a variety of signal processing such as amplification, frequency conversion, phase detection, low-frequency amplification, and filtering.

Meanwhile, the first diagnostic image and the second diagnostic image may be captured from different portions of the object ob. For example, when the object ob is an entire human body, the first diagnostic image may include diagnostic images captured from organs suitable for diagnosis via CT scanning, such as coronary artery, lung, abdomen, and bone) and the second diagnostic image may include diagnostic images captured from organs suitable for diagnosis via MRI, such as brain, muscle, valve, breast, joint, spine, and prostate.

As such, since the first diagnostic image and the second diagnostic image are selectively acquired on the organ basis, costs and time for acquiring an image of the entire human body may be reduced. Furthermore, exposure to radiation may be minimized.

In addition, in order to perform image registration of the first diagnostic image and the second diagnostic image, the object ob may be partitioned and imaged such that the first diagnostic image and the second diagnostic image partially or entirely overlap each.

Referring to FIGS. 1 and 2, the host device 50 may include the input unit 310 and the display unit 320.

The input unit 310 receives an instruction to control operation from a user, generates an electric signal in accordance with the instruction input by the user, and transmits the electric signal to the image processor 340. In this case, the input unit 310 may be implemented using various input devices including a key input device such as a keyboard or a keypad, a touch input device such as a touch sensor or a touch pad, a gesture input device including at least one of a gyro sensor, a geomagnetic sensor, an acceleration sensor, a proximity sensor, and a camera, or a voice input device. In addition, various types of input devices which are currently being developed or planned in the future may also be used.

The display unit 320 serves to display various information related to a radiographic imaging apparatus. The display unit 320 according to an embodiment may display a first diagnostic image, a second diagnostic image, or a registered diagnostic image generated by performing image registration of the first diagnostic image and the second diagnostic image. Particularly, the display unit 320 may include a liquid crystal display (LCD), a light emitting diode (LED), an organic light emitting diode (OLED), an active matrix organic light emitting diode (AMOLED), a flexible display, and a 3D display. The host device 50 may include the storage unit 330 and the image processor 340 as illustrated in FIG. 2.

The storage unit 330 may include non-volatile memories (not shown) to permanently store programs and data to control operation of the image generating apparatus 1 such as magnetic discs and solid state discs and volatile memory (not shown) to temporarily store data generated while controlling operation of the image generating apparatus 1 such as D-RAM and S-RAM.

Particularly, the storage unit 330 may store first diagnostic images received from the first image capturing unit 100, second diagnostic images received from the second image capturing unit 200, or a registered diagnostic image generated by performing image registration of the first and second diagnostic images.

The image processor 340 (e.g. controller) comprises hardware that is configured so as to control the overall operation of the image generating apparatus 1. In this regard, the image processor 340 may be implemented by using a single processor (or microprocessor) or a plurality of processors (microprocessors). Here, as the processor or microprocessor comprises hardware, the processor or microprocessor may be implemented by using an array of a plurality of logic gates or a combination of a universal microprocessor and a memory storing a program executable in the microprocessor. Furthermore, it will be understood by those of ordinary skill in the art that the processor may be implemented by another form of hardware.

In addition, the image processor 340 may extract information for performing image registration from the first diagnostic image and the second diagnostic image and generate a registered diagnostic image based on the extracted registration information. To this end, the image processor 340 may include the following components comprised of hardware: an image adjustment unit 341 to adjust the first and second diagnostic images, a registration information extraction unit 343 to extract registration information from the adjusted diagnostic images, and a registered image generation unit 345 to perform image registration of the first and second diagnostic images based on the registration information. Each of the aforementioned units contains circuitry such as integrated circuits configured for operation.

The image adjustment unit 341 may adjust the first diagnostic image and second diagnostic image. As described above, the first image capturing unit 100 and the second image capturing unit 200 acquire diagnostic images respectively using different methods. Thus, the first diagnostic image and the second diagnostic image need to be adjusted for performing image registration of the first diagnostic image and the second diagnostic image.

The image adjustment unit 341 may adjust and normalize the first diagnostic image and second diagnostic image. Particularly, the image adjustment unit 341 may normalize the first and second diagnostic images according to preset conditions suitable for registration of the first and second diagnostic images. Here, normalization may include adjusting size, accumulation, brightness, color, contrast, or sharpness of the diagnostic image.

For example, for image normalization, the image adjustment unit 341 may enlarge or reduce the first diagnostic image and the second diagnostic image such that the first diagnostic image and the second diagnostic image have the same accumulation and may control brightness such that the first diagnostic image and the second diagnostic image have the same or similar contrast.

With continued reference to FIG. 1, meanwhile, the image adjustment unit 341 may preprocess the first and second diagnostic images, if required, or may generate a cross-sectional image or a 3D image based on the diagnostic images received from the first and second image capturing devices 100 and 200.

The registration information extraction unit 343 extracts required registration information to perform image registration of the first and second diagnostic images. In this case, the registration information refers to information required for registration of the first and second diagnostic images, which can include but is not limited to a patient name, date, etc., The registration information may be extracted by analyzing the first and second images or by analyzing information separately collected while acquiring the first and second images.

The registration information may include distinct points. Here, the distinct points refer to locations or regions used as references for performing image registration of the first and second diagnostic images.

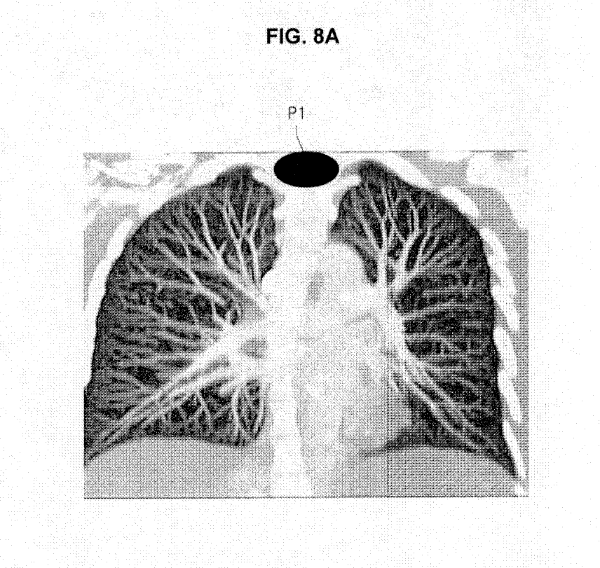

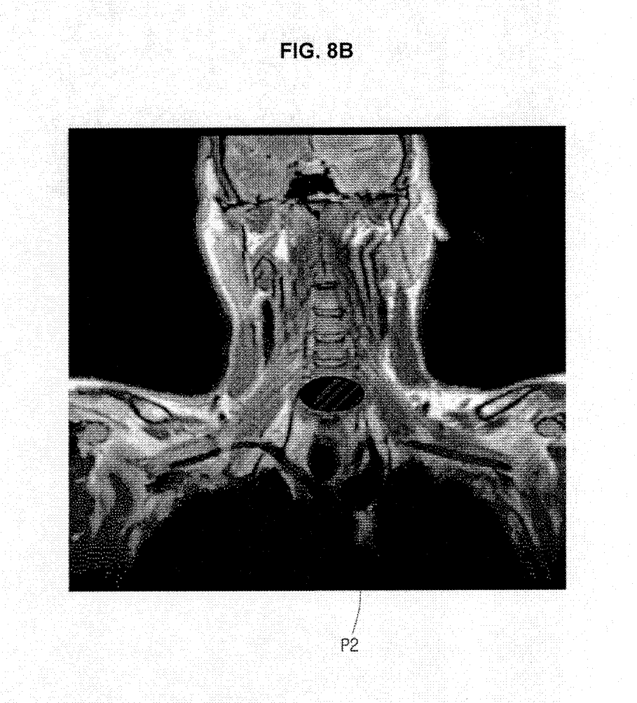

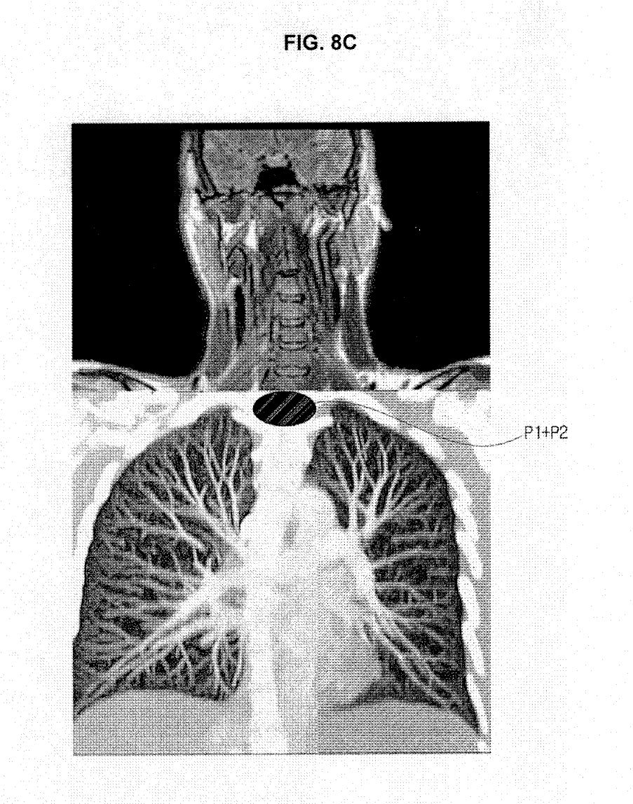

Hereinafter, an example of extraction of the registration information will be described in more detail with reference to FIGS. 8A to 8C. FIGS. 8A to 8C are diagrams for describing an example of image registration. FIG. 8A illustrates a first diagnostic image acquired by the first image capturing unit 100. FIG. 8B illustrates a second image acquired by the second image capturing unit 200. FIG. 8C illustrates a registered diagnostic image according to an embodiment of the present invention.

The registration information extraction unit 343 may extract registration information based on anatomical information. More particularly, the registration information extraction unit 343 may determine locations of the diagnostic images before extracting distinct points. Here, the locations of the diagnostic images may be determined by analyzing the diagnostic images based on the anatomical information or according to image capture conditions when the diagnostic images are acquired. Accordingly, anatomical characteristics that are to be used as references for image registration may be determined based on the analysis results of locations of the first and second diagnostic images.

For example, image information indicating that the first diagnostic image of FIG. 8A is an diagnostic image of a chest, and the second diagnostic image of FIG. 8B is an diagnostic image of a head and neck that may be generated based on schematic shapes of respective portions of the human body created in accordance with anatomical information of the human body. In this regard, the anatomical information refers to various information obtainable by anatomizing the human body, for example, information regarding organs such as location, shape, and size of each organ or information regarding relationship between organs such as positional relationship between the organs and connection between the organs.

In addition, the registration information extraction unit 343 may extract distinct points according to the anatomical information. In other words, the organs of the human body are positioned at relatively constant locations with similar shapes. In addition, the registration information extraction unit 343 may extract distinct points based on the anatomical information of the human body. More particularly, anatomical characteristics, which will be used as references for diagnostic image registration, are determined based on locations of the first and second diagnostic images, and then distinct points corresponding to the anatomical characteristics determined in the first and second diagnostic images may be extracted based on pre-stored anatomical information.

For example, a head and a chest are connected to each other via a spine according to the anatomical information of a human body. The spine is composed of a series of bones including cervical (neck), thoracic (chest/trunk), and lumbar (low back) spines. The cervical spine is located between the head and the chest, and the thoracic spine is located across the chest. Thus, the registration information extraction unit 343 determines the cervical spine and the thoracic spine as anatomical characteristics, and then extracts distinct points from the first diagnostic image by searching for the cervical spine and extracts distinct points from the second diagnostic image by searching for the thoracic spine. As the human body does have variations in size (adults, children) and there can be anomalies (enlarged organs, fused lumbar, etc.) the anatomical characteristics can take into account these differences.

Accordingly, the registration information extraction unit 343 may search for the cervical spine having a sequentially stacked structure at the center of the diagnostic image by analyzing the head diagnostic image and extract an end point P2 of the cervical spine as a distinct point.

In addition, the registration information extraction unit 343 may search for the thoracic spine having a sequentially stacked structure at the center of the diagnostic image by analyzing the chest diagnostic image and extract a starting point P1 of the thoracic spine as a distinct point.

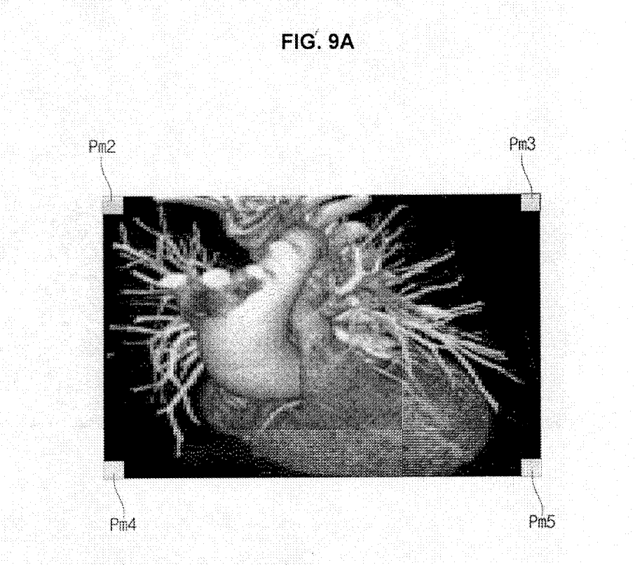

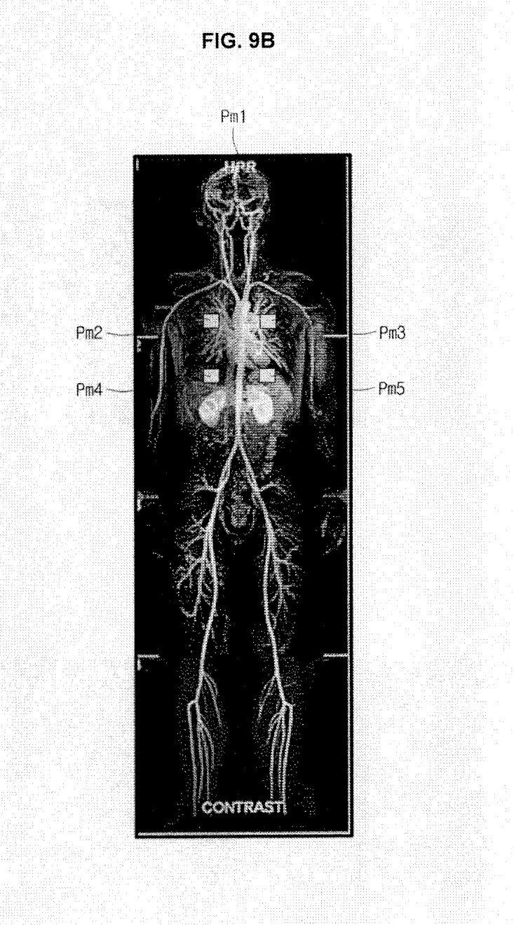

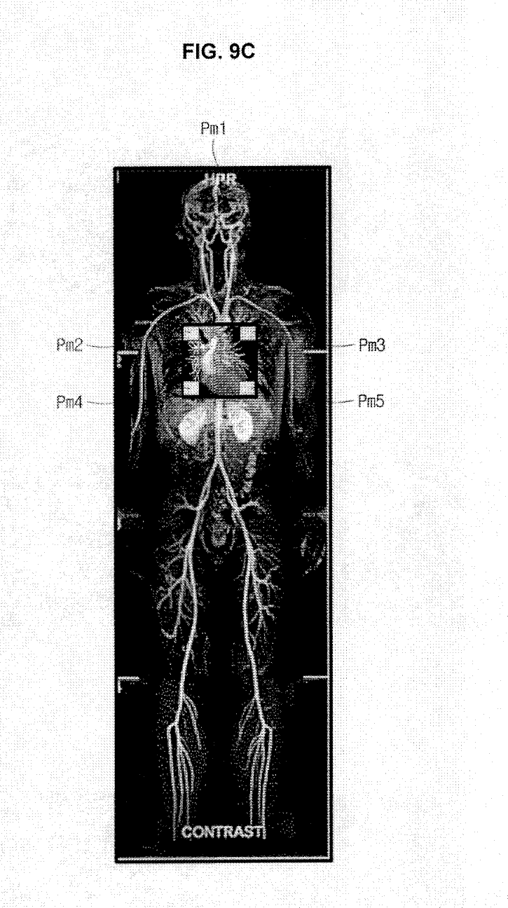

Hereinafter, another example of extraction of the registration information will be described in more detail with reference to FIGS. 9A to 9C.

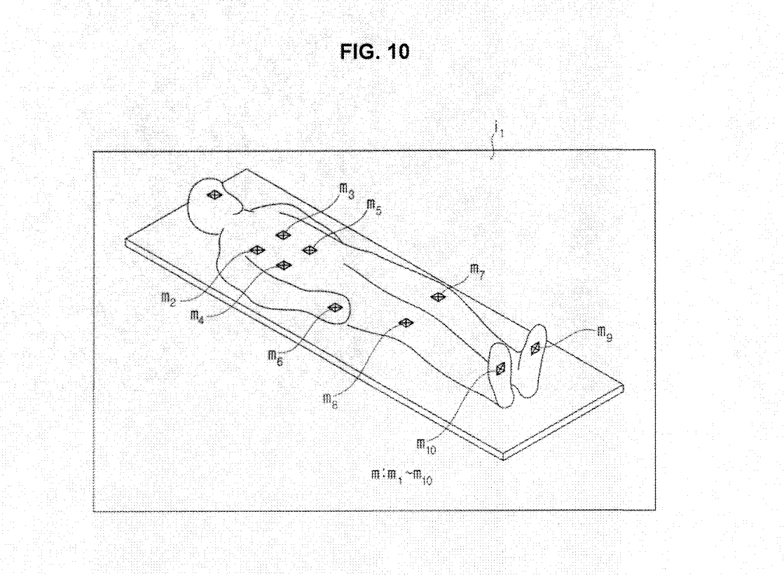

FIGS. 9A to 9C are diagrams for describing another example of diagnostic image registration. FIG. 9A illustrates a first diagnostic image acquired by the first image capturing unit 100. FIG. 9B illustrates a second diagnostic image acquired by the second image capturing unit 200. FIG. 9C illustrates a registered diagnostic image according to an embodiment of the present invention. FIG. 10 is a diagram for describing an example of collecting additional information using markers.

According to another embodiment of the present invention, the registration information extraction unit 343 may generate registration information based on the information collected while acquiring diagnostic images.

More particularly, the registration information may be acquired according to image capture conditions or by attaching a plurality of markers m1 to m10 to the object ob as illustrated in FIGS. 9A to 9C. In this case, the markers m1 to m10 may be attached or bonded to the object ob via an adhesive material or a bonding agent. For example, the markers m1 to m10 may be patches having an adhesive force.

As such, indicators may be created in the first and second diagnostic images by the markers m1 to m10 attached to the object ob. To this end, the markers m1 to m10 may be formed of a material capable of absorbing or reflecting radiation or RF signals.

As illustrated in FIG. 9B, a whole body image may include a plurality of indicators Pm1 to Pm10 created by the plurality of markers m1 to m10. A heart diagnostic image may include a plurality of indicators Pm2 to Pm5 created by markers m2 to m5 corresponding to a chest portion.

Thus, the registration information extraction unit 343 determines that the first diagnostic image of FIG. 9A is a heart diagnostic image and the second diagnostic image of FIG. 9B is a whole body image based on image capture conditions, and then extracts the indicators contained in each diagnostic image as distinct points, thus FIG. 9B shows Pm2, PM3, Pm4 and Pm5 points of FIG. 9A.

The heart diagnostic image and the whole body image may include indicators created by the plurality of markers. Thus, the registration information extraction unit 343 may extract each of the indicators as a distinct point.

The registered image generation unit 345 may generate a registered diagnostic image based on registration information extracted by the registration information extraction unit 343. Particularly, one registered diagnostic image may be generated by using the first and second diagnostic images by combining the distinct points extracted from each diagnostic image.

For example, the head diagnostic image and the chest diagnostic image may be registered based on the distinct points as illustrated in FIG. 8C, or the whole body image and the heart diagnostic image may be registered by using the plurality of distinct points as illustrated in FIG. 9C.

In this regard, the registered diagnostic image generation unit 345 may determine weights of the first and second diagnostic images to be registered and generate a registered diagnostic image in accordance with the determined weights. Particularly, the first and second diagnostic images are captured from a portion of or the entire object ob according to the inner structure or characteristics of the object ob as described above. Thus, the first and second diagnostic images may be captured to be overlapped each other for image registration as illustrated in FIGS. 8A to 9C. Thus, the overlapped portions need to be processed for the diagnostic image registration.

Therefore, the registered image generation unit 345 determines weights of the diagnostic images and processes the overlapped portion of the diagnostic images in accordance with the determined weights. In this regard, the weights may be applied according to the methods of acquiring the first and second diagnostic images or according to settings by a user.

For example, when the overlapped portion is a portion suitable for obtaining a relatively precise image by CT such as coronary artery, lung, abdomen, and bone, a greater weight is applied to the first diagnostic image. When the overlapped portion is a portion suitable for obtaining a relatively precise image by MRI such as brain, muscle, valve, breast, joint, spine, and prostate, a greater weight is applied to the second diagnostic image.

In other words, if the object ob is a human body, the image generating apparatus 1 may acquire diagnostic images by using different methods according to organs or portions to be scanned/diagnosed and generate a diagnosis image by performing image registration of the diagnostic images acquired by using different methods. As described above, by generating a diagnostic image through registration of a plurality of diagnostic images acquired by using different methods, unnecessary exposure to radiation may be prevented, and time and costs for generating the diagnostic image may be reduced.

Furthermore, medical professionals may more efficiently perform diagnosis by registering diagnostic images captured by using different methods. For example, when diagnostic images of various organs of the human body are required, for example, in case of diagnosis of cancer metastasis, the state of the patient may be diagnosed simply and accurately by using the registered diagnostic image.

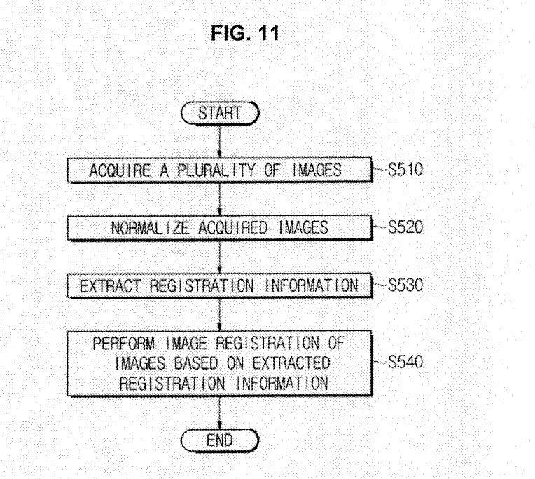

FIG. 11 is a flowchart providing an overview of exemplary tasks for a method of controlling an apparatus for generating an diagnostic image according to an embodiment of the present invention.

With reference to FIG. 11, an image generating apparatus 1 acquires a plurality of diagnostic images of an object ob by using a first image capturing unit 100 and a second image capturing unit 200 (S510). Here, the first diagnostic image capturing unit 100 and the second image capturing unit 200 may acquire diagnostic images by using different methods. A first diagnostic image may be acquired by the first image capturing unit 100, and a second diagnostic image may be acquired by the second image capturing unit 200.

In this regard, the first image capturing unit 100 and the second image capturing unit 200 may acquire diagnostic images of portions of the object ob according to the method of acquiring diagnostic images. For performing image registration of the first diagnostic image and the second diagnostic image, the object ob may be partitioned such that the first diagnostic image and the second diagnostic image partially or entirely overlap each other.

The image generating apparatus 1 normalizes the acquired diagnostic images (S520). Particularly, an image processor 340 may adjust and normalize the first and second diagnostic images. In more detail, the image processor 340 may normalize the first and second diagnostic images according to preset conditions suitable for performing image registration of the first and second diagnostic images.

The image generating apparatus 1 extracts registration information (S530). Here, the registration information may be extracted by analyzing the first and second diagnostic images or by analyzing information separately collected while the first and second diagnostic images are acquired. The registration information may include distinct points. Meanwhile, additional information may also be collected while acquiring diagnostic images for diagnostic image registration. For example, image capture conditions may be collected, or both of the object ob and markers may be captured, as described above.

The image generating apparatus 1 performs image registration of a plurality of diagnostic images based on the extracted distinct points (S540). Here, the image processor 340 determines (assigns) weights of the respective diagnostic images and processes captured portions by overlapping the portions according to the determined weights. In this regard, the weights may be applied according to the methods of acquiring the first and second diagnostic images or according to settings by a user.

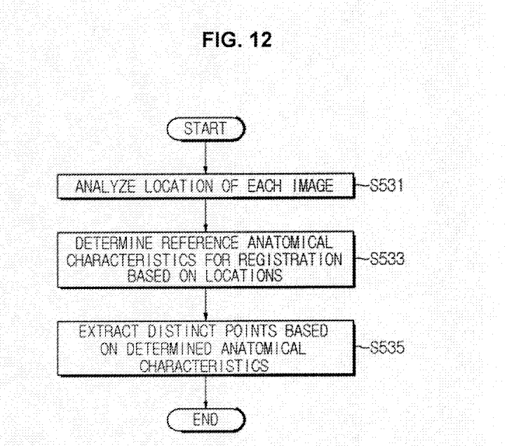

FIG. 12 is a flowchart providing an overview of exemplary tasks for a method of extracting registration information according to an embodiment of the present invention.

The image processor 340 analyzes (identifies) locations of respective diagnostic images (S531). Here, the locations of the diagnostic images may be determined by analyzing the diagnostic images based on the anatomical information or according to image capture conditions when the diagnostic images are acquired. For example, the image processor 340 may analyze the locations of the diagnostic images by comparing pre-stored anatomical information with each diagnostic image or analyze the locations of the diagnostic images based on the image capture conditions obtained while capturing the diagnostic images.

The image processor 340 determines anatomical characteristics which will be used as references for image registration based on the locations of the diagnostic images (S533). The organs of the human body are positioned at relatively constant locations with similar shapes. Thus, anatomical characteristics, to be used as references for image registration, may be determined based on the locations of the diagnostic images.

The image processor 340 extracts distinct points based on the determined anatomical characteristics (S535). More particularly, distinct points corresponding to the anatomical characteristics that are to be used as references for diagnostic image registration, are detected from each diagnostic image based on the pre-stored anatomical information and determined anatomical characteristics.

Meanwhile, embodiments of the present invention can be written as computer programs as machine executable code stored on non-transitory medium and loaded into hardware such as a process, microprocessor or controller (or more than one of the aforementioned) and can be implemented in general-use digital computers that execute the programs using a computer-readable recording medium and by operation of the configuration become special use computers to operate the methods as described herein.

Examples of the computer-readable medium include storage media such as magnetic storage media (e.g., ROMs, floppy discs, or hard discs), optically readable media (e.g., compact disk-read only memories (CD-ROMs), or digital versatile disks (DVDs)), etc.

As is apparent from the above description, costs and time for generating a diagnostic image may be reduced by performing image registration of diagnostic images acquired by using different methods of image capturing.

According to the image generating apparatus and the control method thereof, a clearer and more accurate diagnostic image may be acquired for diagnosis or examination of the human body or the object.

Although a few embodiments of the present invention have been shown and described, it would be appreciated by those skilled in the art that changes may be made in these embodiments without departing from the principles and spirit of the invention, the scope of which is defined in the claims and their equivalents.

The above-described methods according to the present invention can be implemented in hardware, firmware or as software or computer code that is stored on a non-transitory machine readable medium such as a CD ROM, a RAM, thumbnail drive, a floppy disk, a hard disk, or a magneto-optical disk or computer code downloaded over a network originally stored on a remote recording medium or a non-transitory machine readable medium and stored on a local non-transitory recording medium, so that the methods described herein are loaded into hardware such as a general purpose computer, or a special processor or in programmable or dedicated hardware, integrated circuitry including but in no way limited to an ASIC or FPGA. As would be understood in the art, the computer, the processor, microprocessor controller, or the programmable hardware contain circuitry that is typically integrated, and can include memory components, e.g., RAM, ROM, Flash, etc. that may store or receive software or computer code that when accessed and executed by the computer, processor or hardware implement the processing methods described herein. In addition, it would be recognized that when a general purpose computer accesses code for implementing the processing shown herein, the execution of the code transforms the general purpose computer into a special purpose computer for executing the processing shown herein. In addition, an artisan understands and appreciates that a “controller”, “processor” or “microprocessor” constitute hardware in the claimed invention. Under the broadest reasonable interpretation, the appended claims constitute statutory subject matter in compliance with 35 U.S.C. §101 and none of the elements constitute of software per se.

The terms “unit” or “module” as may be used herein is to be understood as constituting or operating in conjunction with hardware such as a circuit, integrated circuit, processor, controller, or microprocessor configured for a certain desired functionality in accordance with statutory subject matter under 35 U.S.C. §101, and such terms do not constitute software per se.

What is claimed is:

1. An apparatus for generating a diagnostic image of an object comprising:
a first image capturing unit configured to acquire a first diagnostic image of the object;
a second diagnostic image capturing unit utilizing a different format of image capturing than the first image capturing unit and being configured to acquire a second diagnostic image of the object different from the first diagnostic image; and
an image processor configured to identify and extract one or more distinct points from both the first diagnostic image and the second diagnostic image and perform image registration of the first and second diagnostic images based on the extracted distinct points.

2. The apparatus according to claim 1, wherein the image processor is configured to extract the distinct points based on pre-stored anatomical information associated with the object.

3. The apparatus according to claim 2, wherein the image processor determines anatomical characteristics to be used as references for image registration by identifying locations of the first and second images, and extracts the distinct points of the first and second diagnostic images by comparing the determined anatomical characteristics with the pre-stored anatomical information.

4. The apparatus according to claim 3, wherein the image processor identifies the locations of the first and second diagnostic images by comparing the pre-stored anatomical information with the first and second diagnostic images.

5. The apparatus according to claim 3, wherein the image processor identifies the locations of the first and second diagnostic images based on image capture conditions for the first and second diagnostic images.

6. The apparatus according to claim 1, wherein the first and second diagnostic images include the object and markers attached to the object, and the image processor extracts the distinct points based on locations of the markers.

7. The apparatus according to claim 1, wherein the image processor normalizes at least one of the first and second diagnostic images.

8. The apparatus according to claim 1, wherein the image processor assigns weights to the first and second diagnostic images, and performs image registration of the first and second diagnostic images by using the weights.

9. The apparatus according to claim 1, wherein the image processor assigns weights according to types of the first image capturing unit and the second image capturing unit, respectively.

10. The apparatus according to claim 1, wherein the first image capturing unit or second image capturing unit comprises at least one apparatus selected from the group consisting of a magnetic resonance imaging (MRI) apparatus, a computed tomography (CT) scanner, a positron emission tomography (PET) scanner, a single photon emission computed tomography (SPECT) scanner, and a mammography apparatus.

11. A method of controlling an apparatus for generating an diagnostic image, the method comprising:
acquiring a first diagnostic image of an object by a first image capturing unit, and acquiring a second diagnostic image different from the first image by a second image capturing unit;
extracting distinct points by an image processor from the first diagnostic image and the second diagnostic image; and
performing image registration in a storage medium the first and second diagnostic images based on the extracted distinct points.

12. The method according to claim 11, wherein the extracting of the distinct points comprises extracting the distinct points based on pre-stored anatomical information.

13. The method according to claim 12, wherein the extracting of the distinct points comprises:
identifying locations of the first and second diagnostic images;
determining anatomical characteristics to be used as references for image registration based on the identified locations of the first and second diagnostic images; and
extracting the distinct points by comparing the determined anatomical characteristics with the pre-stored anatomical information.

14. The method according to claim 13, wherein the locations of the first and second diagnostic images are identified by comparing the pre-stored anatomical information with the first and second diagnostic images.

15. The method according to claim 13, wherein the locations of the first and second diagnostic images are identified based on image capture conditions for the first and second diagnostic images.

16. The method according to claim 11, wherein the acquiring of the first and second diagnostic images comprises acquiring the first diagnostic image and the second diagnostic image by simultaneously capturing diagnostic images of the object and at least one marker, and wherein
the extracting of the distinct points is performed by extracting the distinct points based on locations of the markers.

17. The method according to claim 12, further comprising normalizing at least one of the first and second diagnostic images.

18. The method according to claim 11, wherein the performing image registration of the first and second diagnostic images comprises: assigning weights of the first and second diagnostic images; and processing overlapped portions of the first and second diagnostic images according to the assigned weights.

19. The method according to claim 11, wherein the first or second diagnostic image capturing unit comprises at least one apparatus selected from the group consisting of a magnetic resonance imaging (MRI) apparatus, a computed tomography (CT) scanner, a positron emission tomography (PET) scanner, a single photon emission computed tomography (SPECT) scanner, and a mammography apparatus.

20. The method according to claim 11, where the first image capturing unit and the second image capturing unit communicate via wireless communication to the image processor.
